SEQUENCE LISTING (32 sequences)

SEQ ID NO: 1 - protein, 110 aa, Homo sapiens
GAGCTGCTTGCCTCCCCCCGTTTTTGGCAATGGTAGAACTCACACTGGTGAGGTAACAGGATCCGGTGGTTCTAGACTTGCCAACTATGGGGCGAGGACTCAGCCGGCAC

SEQ ID NO: 2 - protein, 24 aa, Homo sapiens
TTTGGCAATGGTAGAACTCACACT

SEQ ID NO: 3 - protein, 21 aa, Homo sapiens
TGGTTCTAGACTTGCCAACTA

SEQ ID NO: 4 - protein, 14 aa, Homo sapiens
TGAGGTAACAGGAT

SEQ ID NO: 5 - protein, 21 aa, Homo sapiens
AGTGTGAGTTCTACCATTGCC

SEQ ID NO: 6 - protein, 14 aa, Homo sapiens
TTCTACCATTGCCA

SEQ ID NO: 7 - protein, 16 aa, Homo sapiens
AGTTCTACCATTGCCA

SEQ ID NO: 8 - protein, 21 aa, Homo sapiens
TAGTTGGCAAGTCTAGAACCA

SEQ ID NO: 9 - protein, 17 aa, Homo sapiens
GTTGGCAAGTCTAGAAC

SEQ ID NO: 10 - protein, 19 aa, Homo sapiens
TAGTTGGCAAGTCTAGAAC

SEQ ID NO: 11 - protein, 14 aa, Homo sapiens
ATCCTGTTACCTCA

SEQ ID NO: 12 - protein, 12 aa, Homo sapiens
CCTGTTACCTCA

SEQ ID NO: 13 - protein, 12 aa, Homo sapiens
ATCCTGTTACCT

SEQ ID NO: 14 - protein, 23 aa, Homo sapiens
TTGGCAATGGTAGAACTCACACT

SEQ ID NO: 15 - protein, 20 aa, Homo sapiens
GGTTCTAGACTTGCCAACTA

SEQ ID NO: 16 - protein, 23 aa, Homo sapiens
TTTGGCAATGGTAGAACTCACAC

SEQ ID NO: 17 - protein, 20 aa, Homo sapiens
TGGTTCTAGACTTGCCAACT

SEQ ID NO: 18 - protein, 22 aa, Homo sapiens
TGGCAATGGTAGAACTCACACT

SEQ ID NO: 19 - protein, 19 aa, Homo sapiens
GTTCTAGACTTGCCAACTA

SEQ ID NO: 20 - protein, 22 aa, Homo sapiens
TTTGGCAATGGTAGAACTCACA

SEQ ID NO: 21 - protein, 19 aa, Homo sapiens
TGGTTCTAGACTTGCCAAC

SEQ ID NO: 22 - protein, 21 aa, Homo sapiens
GGCAATGGTAGAACTCACACT

SEQ ID NO: 23 - protein, 18 aa, Homo sapiens
TTCTAGACTTGCCAACTA

SEQ ID NO: 24 - protein, 21 aa, Homo sapiens
TTTGGCAATGGTAGAACTCAC

SEQ ID NO: 25 - protein, 18 aa, Homo sapiens
TGGTTCTAGACTTGCCAA

SEQ ID NO: 26 - protein, 16 aa, Homo sapiens
TGGCAATGGTAGAACT

SEQ ID NO: 27 - protein, 17 aa, Homo sapiens
GTTCTAGACTTGCCAAC

SEQ ID NO: 28 - protein, 17 aa, Homo sapiens
CAATGGTAGAACTCACA

SEQ ID NO: 29 - protein, 17 aa, Homo sapiens
GTTCTAGACTTGCCAAC

SEQ ID NO: 30 - protein, 31 aa, Homo sapiens
TTTTTGGCAATGGTAGAACTCACACTGGTGA

SEQ ID NO: 31 - protein, 81 aa, Homo sapiens
CCCCCCGTTTTTGGCAATGGTAGAACTCACACTGGTGAGGTAACAGGATCCGGTGGTTCTAGACTTGCCAACTATGGGGCG

SEQ ID NO: 32 - protein, 97 aa, Homo sapiens
CTTGCCTCCCCCCGTTTTTGGCAATGGTAGAACTCACACTGGTGAGGTAACAGGATCCGGTGGTTCTAGACTTGCCAACTATGGGGCGAGGACTCAG

(19) United States
(12) Patent Application Publication
(10) Pub. No.: US 2014/0309280 A1
(43) Pub. Date: Oct. 16, 2014

(54) ASSAYS FOR MICRO-RNA-182 AS A BIOMARKER FOR MUSCLE ATROPHY AND THERAPEUTIC APPLICATIONS

(71) Applicants: Matthew B. Hudson, Atlanta, GA (US); Stephen Russ Price, Atlanta, GA (US)

(72) Inventors: Matthew B. Hudson, Atlanta, GA (US); Stephen Russ Price, Atlanta, GA (US)

(73) Assignee: EMORY UNIVERSITY, Atlanta, GA (US)

(21) Appl. No.: 14/249,470

(22) Filed: Apr. 10, 2014

Related U.S. Application Data
(60) Provisional application No. 61/811,258, filed on Apr. 12, 2013.

(51) Int. Cl. C12Q 1/68

(52) U.S. Cl. CPC C12Q 1/6883
USPC 514/44 A; 435/6.11; 506/9; 536/24.31

(57) ABSTRACT

In certain embodiments, this disclosure relates to assays for miR-182 and therapeutic applications. In certain embodiments, the disclosure relates to methods of evaluating a state of skeletal muscle atrophy comprising the steps of measuring miR-182 in a sample from a subject wherein decreased quantities of miR-182 indicates an increased state of muscle atrophy in the subject.

### CROSS REFERENCE TO RELATED APPLICATIONS

This application claims priority to U.S. Provisional Application No. 61/811,258 filed Apr. 12, 2013, hereby incorporated by reference in its entirety

### STATEMENT REGARDING FEDERALLY FUNDED RESEARCH

This invention was made with government support under Grant T32DK0076561 awarded by the National Institutes of Health. The government has certain rights in the invention.

### BACKGROUND

Skeletal muscle atrophy is a debilitating condition associated with weakness and fatigue that increases the risk of morbidity and mortality in patients. Skeletal muscle atrophy occurs in response to a variety of conditions including sepsis, glucocorticoid administration, fasting, mechanical ventilation, muscle disuse, chronic kidney disease, diabetes, renal failure, cancer, HIV/AIDS, uremia, and numerous other chronic or systemic diseases.

Skeletal muscle atrophy occurs primarily through increased degradation and removal of muscle proteins by the ubiquitin-proteasome and autophagy-lysosome pathways. An upregulated pathway during conditions of muscle atrophy is the Forkhead box 0 (FoxO) signaling pathway. Activation of FoxO3, a member of the FoxO family, augments the activity of both the ubiquitin-proteasome and autophagy-lysosome pathways, and is sufficient for muscle atrophy. Further, inhibition of FoxO3 attenuates muscle atrophy, and in some models of atrophy promotes hypertrophy. While increased FoxO3 activity is indicative of skeletal muscle atrophy, measurements of FoxO3 directly in muscle is invasive, time consuming, and complex. Measuring muscle size typical entails an invasive muscle biopsy followed by complex and time consuming immunohistochemical staining and analysis of fiber size cross-sectional areas. An estimate of muscle size may be done by MRI, but the process is not as accurate, costly, and often inaccessible. Thus, there is a need for improved methods of detecting muscle atrophy.

MicroRNAs (miRNAs) are a class of endogenous non-coding single-stranded RNA oligonucleotides, of approximately 20-22 nt in length. miRNAs provide a post-transcriptional mechanism by which the levels of specific proteins can be controlled in cells. Interestingly miRNAs appear to be very stable in both tissue and biological fluid. Karolina et al., report identifying four miRNAs, one being miR-182. PLoS One. 2011, 6(8):e22839.

### SUMMARY

In certain embodiments, this disclosure relates to assays for miR-182 and therapeutic applications. In certain embodiments, the disclosure relates to methods of evaluating a state of skeletal muscle atrophy comprising the steps of measuring miR-182 in a sample from a subject wherein decreased quantities of miR-182 indicates an increased state of muscle atrophy in the subject.

In certain embodiments, the disclosure relates to methods of evaluating a state of skeletal muscle atrophy comprising the steps of mixing a probe to miR-182 and a sample from a subject providing a signal; comparing the measured signal to a standard or reference signal thereby quantifying the miR-182-5p and/or miR-182-3p and/or pre-miR-182 stem-loop in the sample wherein decreased quantities of miR-182-5p and/or miR-182-3p and/or pre-miR-182 stem-loop indicates an increased state of muscle atrophy in the subject.

In certain embodiments, the sample is exosomes isolated from biological fluids muscle, tissue, urine, urine exosome, blood, blood exosomes, plasma, bodily fluid, or component thereof.

In certain embodiments, the subject is diagnosed, at risk of, or exhibiting symptoms of cachexia, cancer, sepsis, chronic kidney disease, diabetes, renal failure, a chronic viral infection, HIV/AIDS, uremia, Dejerine Sottas syndrome, multiple sclerosis, tuberculosis, congestive heart failure, COPD, liver disease, muscular dystrophy, rheumatoid arthritis, amyotrophic lateral sclerosis (ALS), spinal muscle atrophy (SMA), or other chronic or systemic disease. In certain embodiments, the subject is on a glucocorticoid therapy, mechanical ventilation, extended ICU stay, fasting, has a cast put on a limb, extended bed rest, or in another state of muscle disuse. In certain embodiments, the subject is a human subject over 50, 60, or 65 years old.

In certain embodiments, assays for measuring miR-182 in the sample entails the use of, but not limited to, fluorescent, radioactive, or marker based hybridization probes, hairpin probes, FRET (fluorescence resonance energy transfer) probes, molecular beacons, nano-flares, template reaction hybridization probes, PCR, quantitative PCR, RNase protection assays, hybridization or sequencing arrays, differential display, northern blotting, or other signal amplification assay.

In certain embodiments, the disclosure relates to probes having a sequence of more than 7, 8, 9, 10, 11, 12, 13, 14, 15 or more nucleotides or nucleobases or continuous nucleotide nucleobases that is the reverse complement of SEQ ID NO: 1, 2, 3 or 4.

In certain embodiments, the disclosure relates to methods of treating muscle atrophy, cachexia, or related disease or condition comprising administering an effective amount of a pharmaceutical composition disclosed herein to a subject in need thereof.

In certain embodiments, the disclosure relates to pharmaceutical composition comprising a nucleotide base comprising a nucleobase miR-182, e.g., wherein the nucleobase polymer is single or double stranded miR-182-5p and/or miR-182-3p and/or pre-miR-182 stem loop, and a pharmaceutically acceptable excipient.

Sanchez et al. report AMPK promotes skeletal muscle autophagy through activation of forkhead FoxO3a and interaction with Ulk1. J Cell Biochem 113: 695-710, 2012.

Sandri et al. report PGC-lalpha protects skeletal muscle from atrophy by suppressing FoxO3 action and atrophy-specific gene transcription. Proc Natl Acad Sci USA 103: 16260-16265, 2006.

Sandri et al. report Foxo transcription factors induce the atrophy-related ubiquitin ligase atrogin-1 and cause skeletal muscle atrophy. Cell 117: 399-412, 2004

Segura et al. report aberrant miR-182 expression promotes melanoma metastasis by repressing FOXO3 and microphthalmia-associated transcription factor. Proc Natl Acad Sci USA 106: 1814-1819, 2009.

Senf et al., report FOXO signaling is required for disuse muscle atrophy and is directly regulated by Hsp70. Am J Physiol Cell Physiol 298: C38-45, 2010.

Hudson et al. report miR-23a is decreased during muscle atrophy by a mechanism that includes calcineurin signaling and exosome-mediated export. Am J Physiol Cell Physiol, 2014, 306: C551-0558.

References cited herein are not an admission of prior art.

### BRIEF DESCRIPTION OF THE FIGURES

FIG. 1 shows data indicating MicroRNA-182 regulates FoxO3 in skeletal muscle cells. A) C2C12 cells were co-transfected with a pMIR-FoxO3-3′UTR luciferase reporter plasmid and either pre-miR miR-182 or control pre-miR microRNA precursors. Luciferase activity is expressed as the percentage of the mean activity with the control microRNA. Shown in the inset is the FoxO3 3′-UTR sequence that contains the more proximal miR-182 interaction site. B-D) C2C12 cells were transfected with either pre-miR miR-182 or control pre-miR microRNA precursors. After 72 hours, endogenous FoxO3 mRNA (B) and protein (C) were quantified. Panel D is a fluorescent microscopy image of C2C12 cells transfected of a Cy3 dye-labeled precursor miRNA to demonstrate transfection efficiency. E) The level of miR-182 was measured in C2C12 cells that were transfected with either pre-miR miR-182 or control pre-miR microRNA precursors.

FIG. 2 shows data indicating changes in miR-182 and FoxO3 expression during atrophy. FoxO3 mRNA (Panels A and C) and miR-182 (Panels B and D) were measured in either cells treated with Dex (1 μM) for 6 h (Panels A and B) or in gastrocnemius muscles of either control or streptozotocin-injected, acutely diabetic rats (Panels C and D). Results are reported as the mean±SEM of the fold-change of the respective control values.

FIG. 3 shows data indicating miR-182 prevents Dex-induced changes in FoxO3 gene targets. C2C12 cells were transfected with either pre-miR miR-182 or control pre-miR microRNA precursors. After 72 hours, some cells were pretreated with Dex (1 μM) for 6 h before measuring the mRNAs for A) atrogin-1, B) LC3, C) ATG12, and D) cathepsin L.

FIG. 4 shows data indicating Dex increases miR-182 in media exosomes from C2C12 myotubes. Fresh differentiation media containing 100 nM Dex or vehicle was added to myotubes for 6 h. Afterwards, RNA was prepared from isolated media exosomes and miR-182, along U6 controls, was measured.

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise.

As used herein, the term “combination with” when used to describe administration with an additional treatment means that the agent may be administered prior to, together with, or after the additional treatment, or a combination thereof.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g., patient) is cured and the condition or disease is eradicated. Rather, embodiments, of the present disclosure also contemplate treatment that merely reduces symptoms, and/or delays conditions or disease progression.

The term “sample” as used herein refers to any biological or chemical mixture for use in the method of the disclosure. The sample can be a biological sample. The biological samples are generally derived from a patient, preferably as a bodily fluid (such as tumor tissue, lymph node, sputum, blood, bone marrow, cerebrospinal fluid, phlegm, saliva, or urine) or cell lysate. The cell lysate can be prepared from a tissue sample (e.g. a tissue sample obtained by biopsy), for example, a tissue sample (e.g. a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy.

As used herein, the term “nucleic acid” is intended to mean a ribonucleic or deoxyribonucleic acid or analog thereof, including a nucleic acid analyte presented in any context; for example, a probe, target or primer. A nucleic acid can include native or non-native bases. In this regard a native deoxyribonucleic acid can have one or more bases selected from the group consisting of adenine, thymine, cytosine or guanine and a ribonucleic acid can have one or more bases selected from the group consisting of uracil, adenine, cytosine or guanine. It will be understood that a deoxyribonucleic acid used in the methods or compositions set forth herein can include uracil bases and a ribonucleic acid can include a thymine base. Exemplary non-native bases that can be included in a nucleic acid, whether having a native backbone or analog structure, include, without limitation, inosine, xathanine, hypoxathanine, isocytosine, isoguanine, 2-aminopurine, 5-methylcytosine, 5-hydroxymethyl cytosine, 2-aminoadenine, 6-methyl adenine, 6-methyl guanine, 2-propyl guanine, 2-propyl adenine, 2-thioLiracil, 2-thiothymine, 2-thiocytosine, 15-halouracil, 15-halocytosine, 5-propynyl uracil, 5-propynyl cytosine, 6-azo uracil, 6-azo cytosine, 6-azo thymine, 5-uracil, 4-thiouracil, 8-halo adenine or guanine, 8-amino adenine or guanine, 8-thiol adenine or guanine, 8-thioalkyl adenine or guanine, 8-hydroxyl adenine or guanine, 5-halo substituted uracil or cytosine, 7-methylguanine, 7-methyladenine, 8-azaguanine, 8-azaadenine, 7-deazaguanine, 7-deazaadenine, 3-deazaguanine, 3-deazaadenine or the like. A particular embodiment can utilize isocytosine and isoguanine in a nucleic acid in order to reduce non-specific hybridization, as generally described in U.S. Pat. No. 5,681,702.

A non-native base used in a nucleic acid can have universal base pairing activity, wherein it is capable of base pairing with any other naturally occurring base. Exemplary bases having universal base pairing activity include 3-nitropyrrole and 5-nitroindole. Other bases that can be used include those that have base pairing activity with a subset of the naturally occurring bases such as inosine, which base-pairs with cytosine, adenine or uracil. Alternatively or additionally, oligonucleotides, nucleotides or nucleosides including the above-described non-native bases can further include reversible blocking groups on the 2′, 3′ or 4′ hydroxyl of the sugar moiety.

The terms “binding,” “binds,” “recognition,” or “recognize” as used herein are meant to include interactions between molecules that may be detected using, for example, a hybridization assay. When hybridization occurs in an antiparallel configuration between two single-stranded polynucleotides, the reaction is called “annealing” and those polynucleotides are described as “complementary”. A double-stranded polynucleotide can be complementary or homologous to another polynucleotide, if hybridization can occur between one of the strands of the first polynucleotide and the second. Complementarity or homology (the degree that one polynucleotide is complementary with another) is quantifiable in terms of the proportion of bases in opposing strands that are expected to form hydrogen bonding with each other, according to generally accepted base-pairing rules.

The term “polymerase chain reaction” (“PCR”) refers to the method of K. B. Mullis U.S. Pat. Nos. 4,683,195, 4,683,202, and 4,965,188, that describe a method for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing, and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are said to be “PCR amplified.”

With PCR, it is possible to amplify a single copy of a specific target sequence to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). Any oligonucleotide or polynucleotide sequence can be amplified with the appropriate set of primer molecules. In particular, the amplified segments created by the PCR process itself are, themselves, efficient templates for subsequent PCR amplifications.

The terms “PCR product,” “PCR fragment,” and “amplification product” refer to the resultant mixture of compounds after two or more cycles of the PCR steps of denaturation, annealing and extension are complete. These terms encompass the case where there has been amplification of one or more segments of one or more target sequences.

The term “amplification reagents” refers to those reagents (deoxyribonucleotide triphosphates, buffer, etc.), needed for amplification except for primers, nucleic acid template, and the amplification enzyme. Typically, amplification reagents along with other reaction components are placed and contained in a reaction vessel (test tube, microwell, etc.).

As used herein, the term “surface” is intended to mean an external part or external layer of a solid support or gel. The solid support can be a rigid solid and optionally can be impermeable to liquids or gases. The solid support can also be a semi-rigid solid, for example, being permeable to liquids or gases. The surface can be in contact with another material such as a gas, liquid, gel, second surface of a similar or different solid support, metal, or coat. The surface, or regions thereof, can be substantially flat. The surface can have surface features such as wells, pits, channels, ridges, raised regions, pegs, posts or the like.

A surface that is used in accordance with the methods set forth herein can be present on any of a variety of substrates. The surface can be located on a substrate or material that provides a solid or semi-solid foundation. Exemplary types of substrate materials include glass, modified glass, functionalized glass, inorganic glasses, microspheres, including inert and/or magnetic particles, plastics, polysaccharides, nylon, nitrocellulose, ceramics, resins, silica, silica-based materials, carbon, metals, an optical fiber or optical fiber bundles, polymers and multiwell (e.g. microtiter) plates. Specific types of exemplary plastics include acrylics, polystyrene, copolymers of styrene and other materials, polypropylene, polyethylene, polybutylene, polyurethanes and Teflon™. Specific types of exemplary silica-based materials include silicon and various forms of modified silicon.

microRNA-182 as a Biomarker of Skeletal Muscle Atrophy

Evidence is presented that miR-182 directly targets FoxO3 via binding to sites in the 3′-UTR in C2C12 cells and that increasing the level of miR-182 decreases both FoxO3 mRNA and protein. miR-182 is decreased during atrophy induced in muscle by diabetes and in cultured myotubes by glucocorticoid administration. Transfection of miR-182 prevents the glucocorticoid-induced increase in FoxO3-responsive genes associated with the ubiquitin-proteasome and autophagy/lysosomal systems. Dex induced a decrease in intracellular miR-182 and increased the incorporation of miR-182 into exosomes released from muscle cells. The data further support the hypothesis that intracellular microRNA levels are regulated through selective packaging of microRNAs into exosomes followed by their release from muscle cells.

In certain embodiments, this disclosure relates to identification of a stable endogenous regulator of FoxO3 activity that may be measured non-invasively providing an indication of FoxO3 activity in the muscle and may be used as a non-invasive biomarker of skeletal muscle atrophy. By identifying a miRNA that regulates FoxO3 endogenously in skeletal muscle, one can determine the level of this miRNA in urine and/or urine exosomes as reflective of the level in muscle during conditions associated with atrophy.

Skeletal muscle atrophy occurs in response to a variety of conditions including chronic kidney disease, diabetes, cancer, and elevated glucocorticoids. MicroRNAs (miR) may play a role in the wasting process. Activation of the Forkhead box 03 (FoxO3) transcription factor causes skeletal muscle atrophy in patients, animals, and cultured cells by increasing the expression of components of the ubiquitin-proteasome and autophagy-lysosome proteolytic systems.

To identify microRNAs that potentially modulate the atrophy process, an in silico target scan analysis was performed and miR-182 was predicted to target FoxO3 mRNA. Using a combination of immunoblot analysis, qPCR, and FoxO3 3′-UTR luciferase reporter genes, miR-182 was confirmed to regulate FoxO3 expression in C2C12 myotubes. Transfection of miR-182 into muscle cells decreased FoxO3 mRNA 30% and FoxO3 protein 67%, and also prevented a glucocorticoid-induced upregulation of multiple FoxO3 gene targets including MAFbx/Atrogin-1, ATG12, Cathepsin L, and LC3. Treatment of C2C12 myotubes with Dexamethasone (Dex) (1 μM, 6 hr) to induce muscle atrophy decreased miR-182 expression by 63% (P<0.05). Similarly, miR-182 was decreased 44% (P<0.05) in the gastrocnemius muscle of rats injected with streptozotocin to induce diabetes compared to controls. Finally, miR-182 was present in exosomes isolated from the media of C2C12 myotubes and Dex increased its abundance. These data indicate that miR-182 is a regulator of FoxO3 expression that participates in the control of atrophy-inducing genes during catabolic diseases.

Probes for miR-182

It is believed that microRNAs are initially transcribed as part of one arm of an ˜80 nucleotide RNA stem-loop (termed a primary microRNA or pri-miRNA). Each pri-miRNA may contain several microRNA precursors, and undergoes processing and cleavage by several proteins and enzymes to form precursor microRNAs (pre-miRNAs). Pre-miRNAs are then exported out of the nucleus and into the cytoplasm where the pre-miRNA hairpin is cleaved by an RNase enzyme (Dicer). Dicer is thought to interact with the 3′ end of the hairpin and cuts away the loop joining the 3′ and 5′ arms resulting in an unstable microRNA duplex, and ultimately resulting in a mature miR-3p and miR-5p.

miR-182 is described in the microRNA database (http://www.mirbase.org/), hereby incorporated by reference. miR-182-5p originates from the 5p end of the miRNA stem loop (pre-miRNA) and miR-182-3p originates from the 3p end. Certain examples disclosed herein utilize mi-R182-5p; however, the methods and compositions disclosed herein may be utilized with probes to miR-182-5p, miR-182-3p, or pre-miR-182.

The sequence of human pri-miR-182 is (SEQ ID NO: 1) GAG CUGCUUGC CUCCCCCCGUUUUUGGCAAUGGUAGAACUCACACUGGUGAGGUAACAGGAUC CGGUGGUUCUAGACUUGCCAACUAUGGGGCGAGGACUCAGCCGGCAC.

The sequence of miR-182-5p in humans is (SEQ ID NO: 2) UUUGGCAAUGGUAGAACUCACACU.

The sequence of miR-182-3p in humans is (SEQ ID NO: 3) UGGUUCUAGACUUGCCAACUA.

Human pre-miR-182 stem-loop structure has the following loop sequence (SEQ ID NO: 4) UGAGGUAACAGGAU.

The term “probe” refers to a molecule capable of hybridizing to a single-stranded nucleic acid target. The probes may target, e.g., comprise a sequence that is the reverse complement of, more than 7, 8, 9, 10, 11, 12, 13, 14, 15, 20, or more nucleotides or nucleobases or continuous nucleotide nucleobases of SEQ ID NO: 1, 2, 3 or 4. The probe may be single stranded nucleic acid or analog containing a sufficiently small number of mismatches, additions, or deletions as long as the probe retains the ability to bind to the target. The probe may be the single stranded tail of a double stranded nucleic acid. The probe may be a part of a loop structure or single stranded tail of a hairpin structure. In certain embodiments, the probe may be less than 500, 200, 100, 50, or 30 nucleotides or nucleobases.

In certain embodiments, the probe contains the reverse complement of SEQ ID NO: 2 or hydrogen bonding mimetic thereof. Example probes include: (SEQ ID NO: 5) AGUGUGAGUUCUACCAUUGCC, (SEQ ID NO: 6) UUCUACCAUUGCCA and (SEQ ID NO: 7) AGUUCUACCAUUGCCA.

In certain embodiments, the probe contains the reverse complement of SEQ ID NO: 3 or hydrogen bonding mimetic thereof. Example probes include: (SEQ ID NO: 8) UAGUUGGCAAGUCUAGAACCA, (SEQ ID NO: 9) GUUGGCAAGUCUAGAAC and (SEQ ID NO: 10) UAGUUGGCAAGUCUAGAAC.

In certain embodiments, the probe contains the reverse complement of SEQ ID NO: 4 or hydrogen bonding mimetic thereof. Example probes include: (SEQ ID NO: 11) AUCCUGUUACCUCA, (SEQ ID NO: 12) CCUGUUACCUCA, and (SEQ ID NO: 13) AUCCUGUUACCU.

In certain embodiments, the disclosure relates to compositions and surfaces comprising probes disclosed herein. In certain embodiments, the probe comprises at least a 7, 8, 9, 10, 11, 12, 13, 14, 15, 16, 17, 18, 19, or 20 nucleotide or nucleobase segment of SEQ ID NO: 5, 6, 7, 8, 9, 10, 11, 12, or 13.

In certain embodiments, the disclosure relates to compositions comprising a probe having at least a 7, 8, or 9 nucleotide or nucleobase segment of SEQ ID NOs: 5, 6, 7, 8, 9, 10, 11, 12, or 13.

In certain embodiments, the disclosure relates to surfaces comprising a probe having at least a 7, 8, or 9 nucleotide or nucleobase segment of SEQ ID NOs: 5, 6, 7, 8, 9, 10, 11, 12, or 13.

The term “nucleobase polymer” refers to a polymer comprising nitrogen containing aromatic or heterocyclic bases that bind to naturally occurring nucleic acids through hydrogen bonding otherwise known as base pairing. A typical nucleobase polymer is a nucleic acid, RNA, DNA, or chemically modified form thereof. A nucleic acid may be single or double stranded or both, e.g., they may contain overhangs. Nucleobase polymers may contain naturally occurring or synthetically modified bases and backbones. In certain embodiments, a nucleobase polymer need not be entirely complementary, e.g., may contain one or more insertions, deletions, or be in a hairpin structure provided that there is sufficient selective binding. With regard to the nucleobases, it is contemplated that the term encompasses isobases, otherwise known as modified bases, e.g., are isoelectronic or have other substitutes configured to mimic naturally occurring hydrogen bonding base-pairs, e.g., U may be substituted for T or other modified nucleobase. Examples of nucleotides with modified adenosine or guanosine include, but are not limited to, hypoxanthine, xanthine, 7-methylguanine Examples of nucleotides with modified cytidine, thymidine, or uridine include 5,6-dihydrouracil, 5-methylcytosine, 5-hydroxymethylcytosine.

In certain embodiments, a nucleobase polymer disclosed herein comprises monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphonamidate, morpholin-2-ylmethanol, (2-(hydroxymethyl)morpholino) (piperazin-1-yl)phosphinate, or peptide nucleic acids or combinations thereof.

Probes can be artificial, synthetic, or naturally occurring oligonucleotides or nucleobase polymers, and typically contain naturally occurring nucleotides, but may contain modified or non-naturally occurring nucleotides such as those having universal bases and isobases. Two particularly useful isobases in probe nucleotides are 2′-deoxy-5-methylisocytidine (iC) and 2′-deoxy-isoguanosine (iG) (see U.S. Pat. No. 6,001,983; No. 6,037,120; No. 6,617,106; and No. 6,977,161). In another embodiment, the probe can contain a nucleotide containing a removable base (such as uracil or 8-oxoguanine) so that treatment by uracil-DNA glycosylase (UDG) or formamidopyrimidine-DNA glycosylase (FPG), can lead to cleavage and degradation of unwanted or excess probes. Probes may permit a limited number of mismatched or degenerate positions as long as they are capable of hybridization.

Methods of Measuring Amounts of miR-182

In certain embodiments, methods of measuring miR-182 in the sample entails the use of, but not limited to, fluorescent, radioactive, or marker based hybridization probes, hairpin probes, FRET (fluorescence resonance energy transfer) probes, molecular beacons, nano-flares, template reaction hybridization probes, PCR, quantitative PCR, RNase protection assays, hybridization or sequencing arrays, differential display, northern blotting, or other signal amplification assay.

In certain embodiments, methods of measuring miR-182 comprise mixing a sample and a probe for miR-182, e.g., miR-182-5p and/or miR-182-3p and/or pre-miR-182 stem-loop under conditions to form a miR-182 probe hybrid, optionally purifying the hybrid, and visualizing by mixing with an intercalating dye.

In certain embodiments, methods of measuring miR-182 entails the use of probes that are radioactive optionally separating the probes from probes that hybridizes to miR-182-5p and/or miR-182-3p and/or pre-miR-182 stem-loop wherein measuring radioactivity indicates the quantity of the probe that hybridizes to miR-182-5p and/or miR-182-3p and/or pre-miR-182 stem-loop in the sample. In certain embodiments, the probe comprises a radioactive isotope of phosphorus ³²P incorporated into the phosphodiester bond in the probe. The probe may be detected by visualizing the hybridized probe via autoradiography or other imaging techniques.

In certain embodiments, methods of measuring miR-182 entails the use of probes that comprise antibody epitopes optionally separate the probes from a probe that hybridizes to miR-182-5p and/or miR-182-3p and/or miR-182 stem-loop wherein measuring antibody binding, e.g., with an antibody conjugated to a fluorescent chromophore or other marker, e.g., enzyme, indicates the quantity of the probe that hybridizes to miR-182-5p and/or miR-182-3p and/or miR-182 stem-loop in the sample. In certain embodiments, the epitope is digoxigenin.

In certain embodiments, the disclosure relates to methods comprising mixing the sample with a composition or surface comprising a probe that hybridizes to miR-182-5p and/or miR-182-3p and/or pre-miR-182 stem-loop and detecting hybridization of the probe to miR-182-5p and/or miR-182-3p and/or pre-miR-182 stem-loop in the sample under conditions such that an amount of miR-182-5p and/or miR-182-3p and/or pre-miR-182 stem-loop is quantified. In certain embodiments, a fluorescent probe sequence or a probe sequence is generated that can be rendered fluorescent later, e.g., a probe conjugated to a ligand that can bind a fluorescent receptor or a probe conjugated to a receptor that can bind a fluorescent ligand or a probe with two oligonucleotide sequences, one that can hybridize to the target and the second that can hybridize to a secondary detection oligonucleotide. The probe and target sequences are then mixed together, and the probe specifically hybridizes to its complementary sequence on the target, i.e., miR-182. If the probe is already fluorescent, it will be possible to directly detect hybridization in the sample. In some instances, an additional step may be needed to visualize the hybridized probe. Hybrids formed between the probes and their targets can be detected using a fluorescent microscope or other visualization device. The intensity of the light signal correlates to the quantity of the target as can be evaluated in light of a standard or reference value using well-known analytical calibration techniques.

In certain embodiments, the probe comprises a FRET acceptor and FRET donor configured such that binding to the probe creates a light signal wherein measuring the intensity of the light signal indicates the quantity of miR-182-5p and/or miR-182-3p and/or pre-miR-182 stem-loop in the sample. Upon exposure to certain wavelengths of electromagnetic radiation, e.g., visible or UV light, energy transfers can occur between two different chromophores, typically referred to as the donor or acceptor, when the chromophores are in short distance, on a molecular scale, to each other. This is referred to as fluorescence resonance energy transfer (FRET). Depending on the chromophores, this can cause a change in fluorescence wavelength emission or quenching. FRET probes are typically designed to have a pair of different chromophores covalently attached to an oligonucleotide sequence that is complementary to a nucleic acid target. In the absence of the target, the fluorescence of the chromophore reporter group is either quenched or generates a unique signal. When the target is added, the oligonucleotide-based probes hybridize to the target and produce a distinctive fluorescence signal or reduction thereof.

Binary probes typically utilized a fluorescence donor and a fluorescence acceptor that are tethered to the ends of two single-stranded oligonucleotides, which are complementary to adjacent regions of a target. When they are not exposed to the target, the two oligonucleotide probes are distributed randomly and separated by large distances, on a molecular scale, in solution. Hybridizing to the target brings the donor and acceptor into close proximity. Upon exciting, the close proximity of the donor to the acceptor results in quenching or other emission change that can be detected.

A molecular beacon refers to a hairpin probe that contains a single-stranded oligonucleotide loop with a chromophore and a quencher attached at its opposite strands of the stem. The central loop sequence is complementary to a target. The chromophore and quencher are arranged at each the stem strands to force the chromophore and quencher to be in close proximity. Hybridization to a target causes the chromophore and the quencher to spatially separate, on a molecular scale, creating a fluorescence change upon photoexcitation.

A scorpion probe is a modification of the molecular beacon wherein the loop sequence in the hairpin contains the target sequence. A single stranded probe sequence to the target or a portion of target, the reverse complement of the target, is added to the 5′end of the hairpin allowing for bind to the target and elongation over the entire length of the target in the presence of amplification reagents, e.g., polymerase and nucleotides. Upon completion of double strand synthesis at the target, the loop sequence hybridizes to the polymerase generated sequence causing separation of the chromophores producing a fluorescent signals.

Nano-flares may be used for detection or nucleic acids. For example, a nanoparticle, e.g., gold nanoparticle or quantum dot, acts as one of the chromophores for FRET. The nanoparticle is conjugated to an oligonucleotide probe containing the target sequence. Hybridized to the probe-nanoparticle conjugate is an oligonucleotide containing the shortened target sequence or portion thereof conjugated to a second chromophore for FRET. The second chromophore on the oligonucleotide is configured to be near the surface, on a molecular scale, of the nanoparticle upon hybridization. When the nanoparticle is exposed to the target, the oligonucleotide containing the shortened target sequence is displaced releasing the second chromophore for detection by photoexcitation.

Templated reaction probes typically utilize two probes that bind next each on a target sequence. The probes create a reactive group that results in a chemical reaction. This reaction can be controlled by effective concentration. The amount of a target sequences can be correlated to the signal resulting from the chemical reaction. Examples include templated photochemical reactions, ester-hydrolysis reactions, nucleophilic substitution reactions, fluorescence signal-generating reactions, photochemical cyclo-addition reactions, and peptide chemical reactions.

In certain embodiments, the disclosure relates to methods of detecting miR-182, particularly pre-miR-182 using PCR and quantitative PCR using primers, e.g., forward and reverse primers are designed to anneal to the stem portion of the hairpin. Polymerase chain reaction (PCR) may be used to quantify the amount of a nucleic acid in a sample. Quantitative PCR is different from standard PCR where the product of the reaction is detected at its end, i.e., by monitoring the reaction progresses in real time. Two typical methods for detection of products in quantitative PCR are non-specific fluorescent dyes that intercalate with any double-stranded nucleic acid, and sequence-specific probes that are labeled with a fluorescent reporter which permits detection only after hybridization of the probe with its complementary target. Detailed methods real-time PCR quantification of precursor and mature microRNA are reported in Schmittgen et al., Methods, 2008, 44(1): 31-38.

In certain embodiments, this disclosure relates to the use of quantitative PCR wherein a detection probe contain a chromophore covalently attached to one end, e.g., 5′-end of the probe, and a quencher at the other end, e.g., 3′-end. The quencher molecule quenches the fluorescence emitted by the chromophore when excited by a source of excitation (FRET). Hybridization probes target a region within a sequence to be amplified by a set of PCR primers. Taq polymerase extends the hybridization primer and synthesizes the nascent strand. The 5′ to 3′ exonuclease activity of the polymerase degrades the detection probe resulting in fluorescence due to separation of the donor and acceptor. The fluorescence signal correlates proportionally to the amount of nucleic acid.

In certain embodiments, quantitative PCR is performed by using a stem-loop primer containing a single stranded tail that hybridizes to one end, e.g., 3′ end, of a miRNA, such as miR-182-5p or miR-182-3p. The stem-loop primer initially adds a polynucleotide sequence within the stem-loop to the end of microRNA resulting in a longer sequence creating more room for forward and reverse primer amplification and annealing of the detection probe, e.g., the added sequence in the stem-loop primer contains the template for a reverse primer in the loop sequence of the stem-loop primer and optionally a target sequence for all or a portion of the detection probe in the stem sequence of the stem-loop primer.

Northern blotting may be used to visualize the amount of a target nucleic acid in a sample. A mixture of nucleic acids are separated by gel electrophoresis, transferred to a solid matrix (such as a nylon membrane), and mixed with target probes to provide qualitative or quantitative information of nucleic acid levels. In certain embodiments, miR-182-5p and/or miR-182-3p and/or pre-miR-182 stem-loop is quantified by performing electrophoresis under conditions that separates the probe hybridized to miR-182-5p, transferring probe hybridized to miR-182-5p and/or miR-182-3p and/or pre-miR-182 stem-loop to a medium, visualizing the probe hybridized to miR-182-5p and/or miR-182-3p and/or pre-miR-182 stem-loop on the medium.

In certain embodiments, nuclease protection assays, e.g., ribonuclease protection assays (RPAs) and S1 nuclease assays may be used for the quantitation of miR-182-5p. Probes, e.g., radiolabeled, are mixed with the sample to hybridize to the target. Remaining hybridized probes are removed by digestion with a mixture of nucleases followed by steps in which the nucleases are inactivated and probe-target hybrids are precipitated. These products are separated, e.g., on a denaturing polyacrylamide gel and are visualized by autoradiography. If non-isotopic probes are used, samples may be visualized by transferring the gel to a membrane and performing secondary detection with antibodies or other appropriate binding agent.

In certain embodiments, this disclosure relates to methods disclosed herein wherein the probe is radioactive further comprising the steps of measuring radioactivity and correlating radioactivity to the quantity of the probe that hybridizes to miR-182-5p and/or miR-182-3p and/or pre-miR-182 stem-loop in the sample.

In certain embodiments, this disclosure relates to methods disclosed herein wherein the probe is radioactive further comprising the step of mixing the composition with nucleases that specifically cleave single-stranded nucleic acids but do not cleave double-stranded nucleic acids

In certain embodiments, methods disclosed herein include the steps of separating the probes from a probe that hybridizes to miR-182-5p and/or miR-182-3p and/or pre-miR-182 stem-loop by mixing with a composition comprising nucleases that specifically cleave single-stranded nucleic acids but do not cleave double-stranded nucleic acids.

In certain embodiments, any of the methods disclosed herein may utilize a surface, wherein the surface comprises a probe conjugated to the surface. In certain embodiments, the surface is an array, bead, or nanoparticle.

In certain embodiment, for any of the methods disclosed herein, measuring is under conditions that a signal is produced and comparing the signal to a standard or reference value indicating the quantity of miR-182-5p and/or miR-182-3p and/or pre-miR-182 stem-loop in the sample.

In certain embodiments, the disclosure provides for kits for carrying out any of the methods described herein. Kits of the disclosure may comprise at least one probe specific for miR-182, and may further include reagents, solid surfaces, primers, or instructions for carrying out the assay method. Kits may also comprise reference samples, that is, useful for comparing signals to reference values.

The instructions relating to the use of the kit for carrying out the disclosure generally describe how the contents of the kit are used to carry out the methods of the disclosure. Instructions may include information as sample requirements (e.g., form, pre-assay processing, and size), steps necessary to measure the miR-182, and interpretation of results.

Instructions supplied in the kits of the disclosure are typically written instructions on a label or package insert (e.g., a paper sheet included in the kit), but machine-readable instructions (e.g., instructions carried on a magnetic or optical storage disk) are also acceptable. In certain embodiments, machine-readable instructions comprise software for a programmable digital computer for comparing the measured values obtained using the reagents included in the kit.

Uses of miR-182 in Treatment or Prevention of Muscle Wasting

In certain embodiments, the disclosure relates to methods of treating muscle wasting, cachexia or related disease or condition comprising administering an effective amount of a pharmaceutical composition comprising a nucleobase miR-182 as disclosed herein to a subject in need thereof.

In certain embodiments, the subject is diagnosed, at risk of or exhibiting symptoms of cachexia, sepsis, chronic kidney disease, diabetes, renal failure, cancer, a chronic viral infection, HIV/AIDS, uremia, Dejerine Sottas syndrome, multiple sclerosis, tuberculosis, congestive heart failure, COPD, liver disease, muscular dystrophy, rheumatoid arthritis, amyotrophic lateral sclerosis (ALS), or other chronic or systemic disease.

In certain embodiments, the subject is diagnosed, at risk of or exhibiting symptoms of cachexia and the pharmaceutical composition comprising miR-182 is optionally administered in combination with an omega-3 fatty acid such as eicosapentaenoic acid, anti-cancer agent, immunosuppressive agent, anti-inflammatory agent, humanized monoclonal antibody against interleukin-6 such as tocilizumab.

### Pharmaceutical Compositions

In certain embodiments, the disclosure relates to pharmaceutical compositions comprising a nucleotide base polymer comprising a nucleobase miR-182 and a pharmaceutically acceptable excipient.

In certain embodiments, the disclosure relates to pharmaceutical compositions comprising nucleobase polymers with SEQ ID NO: 1 or fragment thereof wherein U may be T. In certain embodiments, the fragment is greater than 5, 10, 15, or 20 nucleotides or nucleobases.

In certain embodiments, the disclosure relates to pharmaceutical compositions comprising a double or single stranded nucleotide base polymer comprising or consisting essentially of:

(SEQ ID NO: 1) GAGCUGCUUGCCUCCCCCCGUUUUUGGCAAUGGUAGAACU CACACUGGUGAGGUAACAGGAUCCGGUGGUUCUAGACUUGCCAACUAUGGGG CGAGGACUCAGCCGGCAC,

(SEQ ID NO: 2) UUUGGCAAUGGUAGAACUCACACU or

(SEQ ID NO: 3) UGGUUCUAGACUUGCCAACUA, or fragments thereof wherein U may be T, optionally comprising isobases.

In certain embodiments, the disclosure relates to pharmaceutical compositions comprising a double or single stranded nucleotide base polymer comprising or consisting essentially of:

(SEQ ID NO: 14) UUGGCAAUGGUAGAACUCACACU,

(SEQ ID NO: 15) GGUUCUAGACUUGCCAACUA,

(SEQ ID NO: 16) UUUGGCAAUGGUAGAACUCACAC, or

(SEQ ID NO: 17) UGGUUCUAGACUUGCCAACU, wherein U may be T, optionally comprising isobases.

In certain embodiments, the disclosure relates to pharmaceutical compositions comprising a double or single stranded nucleotide base polymer comprising or consisting essentially of:

(SEQ ID NO: 18) UGGCAAUGGUAGAACUCACACU,

(SEQ ID NO: 19) GUUCUAGACUUGCCAACUA,

(SEQ ID NO: 20) UUUGGCAAUGGUAGAACUCACA, or

(SEQ ID NO: 21) UGGUUCUAGACUUGCCAAC, wherein U may be T, optionally comprising isobases.

In certain embodiments, the disclosure relates to pharmaceutical compositions comprising a double or single stranded nucleotide base polymer comprising or consisting essentially of:

(SEQ ID NO: 22) GGCAAUGGUAGAACUCACACU

(SEQ ID NO: 23) UUCUAGACUUGCCAACUA,

(SEQ ID NO: 24) UUUGGCAAUGGUAGAACUCAC, or

(SEQ ID NO: 25) UGGUUCUAGACUUGCCAA, wherein U may be T, optionally comprising isobases.

In certain embodiments, the disclosure relates to pharmaceutical compositions comprising a double or single stranded nucleotide base polymer comprising or consisting essentially of:

(SEQ ID NO: 26) UGGCAAUGGUAGAACU,

(SEQ ID NO: 27) GUUCUAGACUUGCCAAC,

(SEQ ID NO: 28) CAAUGGUAGAACUCACA, or

(SEQ ID NO: 29) GUUCUAGACUUGCCAAC, wherein U may be T, optionally containing isobases.

In certain embodiments, the disclosure relates to pharmaceutical compositions comprising a double or single stranded nucleotide base polymer comprising or consisting essentially of:

(SEQ ID NO: 30) UUUUUGGCAAUGGUAGAACUCACACUGGUGA GGUAACAGGAUCCGGUGGUUCUAGACUUGCCAACUA,

(SEQ ID NO:31) CCCCCCGUUUUUGGCAAUGGUAGAACUCACA CUGGUGAGGUAACAGGAUCCGGUGGUUCUAGACUUGCCAACUAUGGGGCG, or (SEQ ID NO: 32) CUUGCCUCCCCCCGUUUUUGGCAAUGGUAGAA CUCACACUGGUGAGGUAACAGGAUCCGGUGGUUCUAGACUUGCCAACUAUGG GGCGAGGACUCAG, wherein U may be T, optionally comprising isobases.

In certain embodiments, the double stranded RNA that is 3′ end capped with one, two, or more thymidine nucleotides and/or the passenger strand of the RNA comprises 5′ end polyphophosphate.

In certain embodiments, the disclosure relates to compounds, compositions, and methods disclosed herein using nucleobase polymers. In particular, the instant disclosure features nucleic acid molecules, such as short interfering short interfering RNA (siRNA), double-stranded RNA (dsRNA), micro-RNA (miRNA), and short hairpin RNA (shRNA) molecules.

In certain embodiments, the nucleobase polymer comprises monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphonamidate, morpholin-2-ylmethanol, (2-(hydroxymethyl)morpholino) (piperazin-1-yl)phosphinate, or peptide nucleic acids or combinations thereof.

In certain embodiments, the pharmaceutical composition is in the form of a pill, capsule, tablet, gel, or aqueous buffer comprising a saccharide.

In certain embodiments, the pharmaceutical composition comprising a nucleobase polymer can comprise a delivery vehicle, including liposomes, for administration to a subject, carriers and diluents and their salts, and/or can be present in pharmaceutically acceptable formulations. U.S. Pat. No. 6,395,713 and U.S. Pat. No. 5,616,490 further describe general methods for delivery of nucleic acid molecules. Nucleobase polymers can be administered to cells by a variety of methods known to those of skill in the art, including, but not restricted to, encapsulation in liposomes, by iontophoresis, or by incorporation into other vehicles, such as biodegradable polymers, hydrogels, cyclodextrins (see for example U.S. Pat. No. 7,141,540 and U.S. Pat. No. 7,060,498), poly(lactic-co-glycolic)acid (PLGA) and PLCA microspheres (see for example U.S. Pat. No. 6,447,796), biodegradable nanocapsules, and bioadhesive microspheres, or by proteinaceous vectors (U.S. Pat. No. 7,067,632). In another embodiment, the nucleobase polymers can also be formulated or complexed with polyethyleneimine and derivatives thereof, such as polyethyleneimine-polyethyleneglycol-N-acetylgalactosamine (PEI-PEG-GAL) or polyethyleneimine-polyethyleneglycol-tri-N-acetylgalactosamine (PEI-PEG-triGAL) derivatives.

In one embodiment, a nucleobase polymer is complexed with membrane disruptive agents such as those described in U.S. Pat. No. 6,835,393. In another embodiment, the membrane disruptive agent or agents and nucleobase polymers are also complexed with a cationic lipid or helper lipid molecule, such as those lipids described in U.S. Pat. No. 6,235,310.

Embodiments of the disclosure feature a pharmaceutical composition comprising one or more nucleobase polymers in an acceptable carrier, such as a stabilizer, buffer, and the like. The nucleobase polymers or oligonucleotides can be administered and introduced into a subject by any standard means, with or without stabilizers, buffers, and the like, to form a pharmaceutical composition. When it is desired to use a liposome delivery mechanism, standard protocols for formation of liposomes can be followed. The compositions can also be formulated and used as tablets, capsules or elixirs for oral administration, suppositories for rectal administration, sterile solutions, suspensions for administration by injection, and the other compositions known in the art.

Embodiments of the disclosure also feature the use of the composition comprising surface-modified liposomes containing poly (ethylene glycol) lipids (PEG-modified, or long-circulating liposomes or stealth liposomes). These formulations offer a method for increasing the circulation and accumulation of in target tissues. The long-circulating liposomes enhance the pharmacokinetics and pharmacodynamics of DNA and RNA. See U.S. Pat. No. 5,820,873 and U.S. Pat. No. 5,753,613. Long-circulating liposomes are also likely to protect from nuclease degradation.

Compositions intended for oral use can be prepared according to any method known to the art for the manufacture of pharmaceutical compositions and such compositions can contain one or more such sweetening agents, flavoring agents, coloring agents or preservative agents in order to provide pharmaceutically elegant and palatable preparations. Tablets contain the active ingredient in admixture with non-toxic pharmaceutically acceptable excipients that are suitable for the manufacture of tablets. These excipients can be, for example, inert diluents; such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets can be uncoated or they can be coated by known techniques. In some cases such coatings can be prepared by known techniques to delay disintegration and absorption in the gastrointestinal tract and thereby provide a sustained action over a longer period. For example, a time delay material such as glyceryl monosterate or glyceryl distearate can be employed.

Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin or olive oil.

Aqueous suspensions contain the active materials in a mixture with excipients suitable for the manufacture of aqueous suspensions. Such excipients are suspending agents, for example sodium carboxymethylcellulose, methylcellulose, hydropropyl-methylcellulose, sodium alginate, polyvinylpyrrolidone, gum tragacanth and gum acacia; dispersing or wetting agents can be a naturally-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions can also contain one or more preservatives, for example ethyl, or n-propyl p-hydroxybenzoate, one or more coloring agents, one or more flavoring agents, and one or more sweetening agents, such as sucrose or saccharin.

Oily suspensions can be formulated by suspending the active ingredients in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents and flavoring agents can be added to provide palatable oral preparations. These compositions can be preserved by the addition of an anti-oxidant such as ascorbic acid

Dispersible powders and granules suitable for preparation of an aqueous suspension by the addition of water provide the active ingredient in admixture with a dispersing or wetting agent, suspending agent and one or more preservatives. Suitable dispersing or wetting agents or suspending agents are exemplified by those already mentioned above. Additional excipients, for example sweetening, flavoring and coloring agents, can also be present.

The compositions will generally be administered in an “effective amount”, by which is meant any amount of particles that, upon suitable administration, is sufficient to achieve the desired therapeutic or prophylactic effect in the subject to which it is administered. Usually, depending on the condition to be prevented or treated and the route of administration, such an effective amount will usually be between 0.01 to 100 mg of nucleobase polymer per kilogram body weight of the patient per day, more often between 0.01 and 50 mg, such as between 0.1 and 2.5 mg, for example about 0.1, 0.5, 1, 5, 10, 2, 5, 10, 15, 20 or 25 mg of nucleobase polymer, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated.

### Synthesis of Nucleobases Polymers

One synthesizes oligonucleotides (e.g., certain modified oligonucleotides or portions of oligonucleotides) using protocols known in the art as, for example, described in U.S. Pat. No. 6,001,311. The synthesis of oligonucleotides makes use of common nucleic acid protecting and coupling groups, such as dimethoxytrityl at the 5′-end and phosphoramidites at the 3′-end. In a non-limiting example, small scale syntheses are conducted on a 394 Applied Biosystems, Inc. synthesizer using a 0.2 micro mol scale protocol with a 2.5 min coupling step for 2′-O-methylated nucleotides and a 45 second coupling step for 2′-deoxy nucleotides or 2′-deoxy-2′-fluoro nucleotides. Alternatively, syntheses at the 0.2 micro mol scale can be performed on a 96-well plate synthesizer, such as the instrument produced by Protogene (Palo Alto, Calif.) with minimal modification to the cycle. A 33-fold excess of 2′-O-methyl phosphoramidite and a 105-fold excess of S-ethyl tetrazole can be used in each coupling cycle of 2′-O-methyl residues relative to polymer-bound 5′-hydroxyl. A 22-fold excess of deoxy phosphoramidite and a 70-fold excess of S-ethyl tetrazole mop can be used in each coupling cycle of deoxy residues relative to polymer-bound 5′-hydroxyl. Other oligonucleotide synthesis reagents for the 394 Applied Biosystems, Inc. synthesizer include the following: detritylation solution is 3% TCA in methylene chloride (ABI); capping is performed with 16% N-methyl imidazole in THF (ABI) and 10% acetic anhydride/10% 2,6-lutidine in THF (ABI); and oxidation solution is 16.9 mM I₂, 49 mM pyridine, 9% water in THF (PerSeptive Biosystems, Inc.). S-Ethyltetrazole solution (0.25 M in acetonitrile) is made up from the solid obtained from American International Chemical, Inc. Alternately, for the introduction of phosphorothioate linkages, Beaucage reagent (3H-1,2-benzodithiol-3-one 1,1-dioxide, 0.05 M in acetonitrile) is used.

Deprotection of the DNA-based oligonucleotides is performed as follows: the polymer-bound trityl-on oligonucleotide is transferred to a 4 mL glass screw top vial and suspended in a solution of 40% aqueous methylamine (1 mL) at 65 degrees for 10 minutes. After cooling to −20 degrees, the supernatant is removed from the polymer support. The support is washed three times with 1.0 mL of EtOH:MeCN:H₂O/3:1:1, vortexed and the supernatant is then added to the first supernatant. The combined supernatants, containing the oligonucleotide, are dried.

Alternatively, the nucleic acid molecules can be synthesized separately and joined together post-synthetically, for example, by ligation or by hybridization following synthesis and/or deprotection.

Nucleic acids can also be assembled from two distinct nucleic acid strands or fragments wherein one fragment includes the sense region and the second fragment includes the antisense region of the RNA molecule.

The nucleic acid molecules can be modified extensively to enhance stability by modification with nuclease resistant groups, for example, 2′-amino, 2′-O-allyl, 2′-fluoro, 2′-O-methyl, 2′-H). Constructs can be purified by gel electrophoresis using general methods or can be purified by high pressure liquid chromatography and re-suspended in water.

Chemically synthesizing nucleic acid molecules with modifications (base, sugar and/or phosphate) can prevent their degradation by serum ribonucleases, which can increase their potency. See e.g., U.S. Pat. No. 5,652,094, U.S. Pat. No. 5,334,711, and U.S. Pat. No. 6,300,074. All of the above references describe various chemical modifications that can be made to the base, phosphate and/or sugar moieties of the nucleic acid molecules described herein. Modifications that enhance their efficacy in cells, and removal of bases from nucleic acid molecules to shorten oligonucleotide synthesis times and reduce chemical requirements are desired.

In one embodiment, nucleic acid molecules include one or more (e.g., about 1, 2, 3, 4, 5, 6, 7, 8, 9, 10, or more) G-clamp nucleotides. A G-clamp is a tricyclic aminoethyl-phenoxazine 2′-deoxycytidine or analogue. See Lin &. Matteucci, J Am Chem Soc, 1998, 120, 8531-8532; Flanagan, et al., Proc Nat Acad Sci USA, 1999, 96, 3513-3518; and Maier, et al., Biochemistry, 2002, 41, 1323-1327. A single G-clamp analog substitution within an oligonucleotide can result in substantially enhanced helical thermal stability and mismatch discrimination when hybridized to complementary oligonucleotides. The inclusion of such nucleotides in nucleic acid molecules results in both enhanced affinity and specificity to nucleic acid targets, complementary sequences, or template strands.

In another embodiment, nucleic acid molecules include one or more (e.g., about 1, 2, 3, 4, 5, 6, 7, 8, 9, 10, or more) LNA “locked nucleic acid” nucleotides (see for example U.S. Pat. No. 6,639,059, U.S. Pat. No. 6,670,461, U.S. Pat. No. 7,053,207).

In another embodiment, the disclosure features conjugates and/or complexes of nucleobase polymers. Such conjugates and/or complexes can be used to facilitate delivery of polymers into a biological system, such as a cell. Contemplated conjugates include those with cell penetrating peptide. The conjugates and complexes provided may impart therapeutic activity by transferring therapeutic compounds across cellular membranes, altering the pharmacokinetics, and/or modulating the localization of nucleic acid molecules. In general, the transporters described are designed to be used either individually or as part of a multi-component system, with or without degradable linkers. These compounds are expected to improve delivery and/or localization of nucleic acid molecules into a number of cell types originating from different tissues, in the presence or absence of serum (see U.S. Pat. No. 5,854,038). Conjugates of the molecules described herein can be attached to biologically active molecules via linkers that are biodegradable, such as biodegradable nucleic acid linker molecules.

In another aspect a nucleobase polymers comprises one or more 5′ and/or a 3′-cap structure, for example on only the sense strand, the antisense strand, or both strands.

A “cap structure” refers to chemical modifications, which have been incorporated at either terminus of the oligonucleotide. See, for example, Adamic et al., U.S. Pat. No. 5,998,203. These terminal modifications protect the nucleic acid molecule from exonuclease degradation, and may help in delivery and/or localization within a cell. The cap may be present at the 5′-terminus (5′-cap) or at the 3′-terminal (3′-cap) or may be present on both termini. In non-limiting examples, the 5′-cap includes, but is not limited to, glyceryl, inverted deoxy abasic residue (moiety); 4′,5′-methylene nucleotide; 1-(beta-D-erythrofuranosyl) nucleotide, 4′-thio nucleotide; carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety.

Non-limiting examples of the 3′-cap include, but are not limited to, glyceryl, inverted deoxy abasic residue (moiety), 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non-bridging methylphosphonate and 5′-mercapto moieties (for more details see Beaucage and Iyer, 1993, Tetrahedron 49, 1925).

In one embodiment, the disclosure features modified nucleobase polymer, with phosphate backbone modifications comprising one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions.

### EXAMPLES

### MicroRNA-182 Regulates FoxO3 in Skeletal Muscle Cells

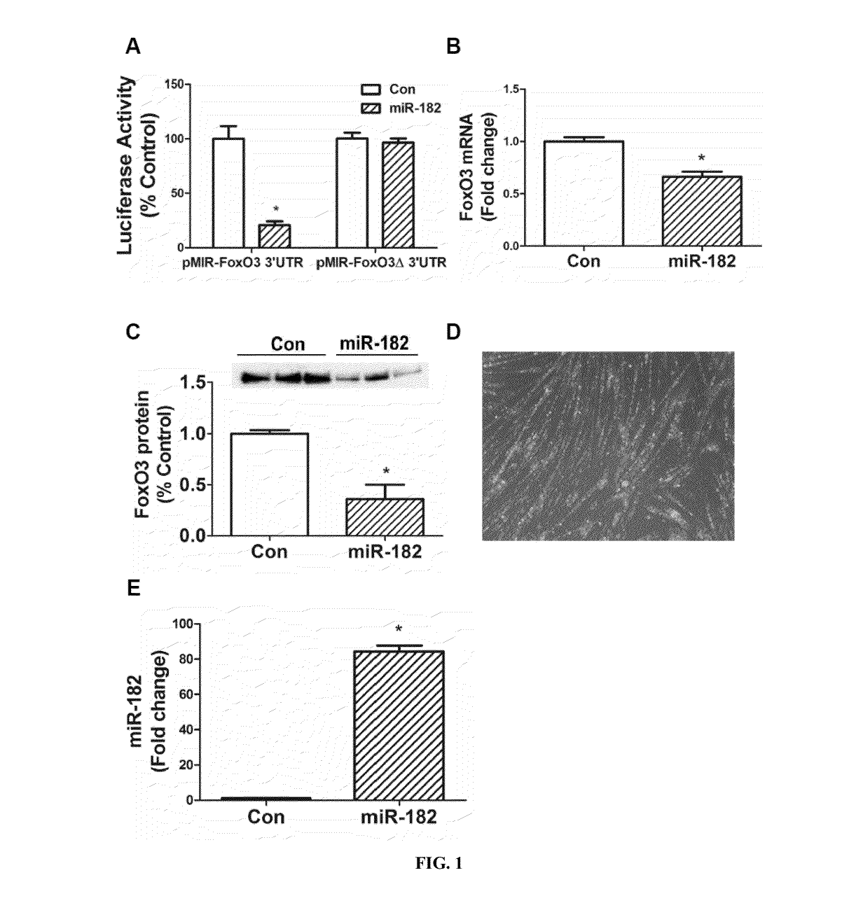

An in silico analysis to identify candidate microRNAs that potentially regulate FoxO3 was performed with TargetScan (www.targetscan.org) and several microRNAs were identified. miR-182 was tested because there are two predicted target sequences in the 3′-UTR of FoxO3. To confirm that miR-182 targets FoxO3 in C2C12 myotubes, myoblasts were transfected with FoxO3-3′-UTR luciferase reporter plasmids plus miR-182 or a scrambled control microRNA and differentiated into myotubes before measuring luciferase activity. Compared to the activity in cells transfected with the control pMIR reporter plasmid, miR-182 significantly reduced the activity of pMIR-FoxO3-3′-UTR but not of the mutated reporter pMIR-FoxO3×3′-UTR (FIG. 1A). Whether miR-182 regulates FoxO3 expression in muscle cells was tested by measuring the levels of FoxO3 mRNA and protein in cells transfected with the miR-182 precursor. miR-182 significantly reduced FoxO3 mRNA by 30% (FIG. 1B) and FoxO3 protein by 67% (FIG. 1C). Efficient transfection of the miR-182 precursor was verified using a Cy3 labeled microRNA precursor (FIG. 1D), and by confirming an increase in the level of intracellular miR-182 by qPCR (FIG. 1E).

miR-182 is Suppressed During Muscle Atrophy.

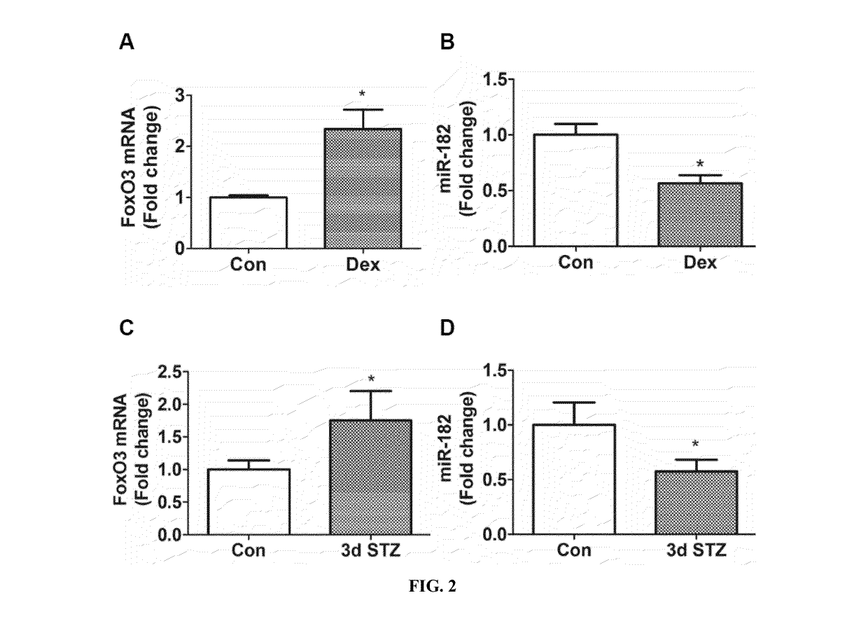

FoxO3 expression is frequently linked to atrophy-inducing conditions indicating that miR-182 may be reduced under these conditions. To test this, C2C12 myotubes were treated with dexamethasone (Dex), a synthetic glucocorticoid, which induces atrophy, in part, by increasing FoxO3 activity in C2C12 myotubes. After 6 hours, FoxO3 mRNA was increased 133% (FIG. 2A). Simultaneously, the amount of intracellular miR-182 decreased 44% (FIG. 2B).

Whether there is a similar relationship between FoxO3 and miR-182 expression was investigated in vivo in skeletal muscle during atrophy. STZ-induced diabetes mellitus causes muscle atrophy and responses (e.g., increased atrogin-1 expression) that are consistent with FoxO3 activation. Therefore, FoxO3 expression was evaluated during STZ-induced diabetes mellitus by measuring FoxO3 mRNA. FoxO3 mRNA was increased 75% (FIG. 2D) whereas the level of miR-182 was decreased 43% (P<0.05) (FIG. 2D). This indicates that an inverse relationship exists between FoxO3 and miR-182 in vitro in cultured muscle cells and in vivo in response to atrophy-inducing conditions.

miR-182 can Prevent Atrophy-Associated Gene Expression

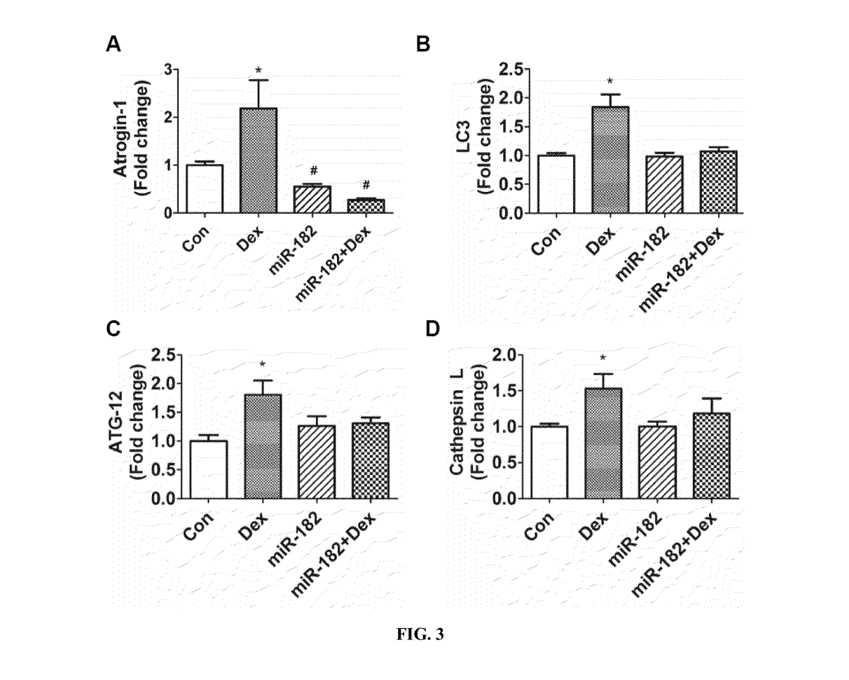

Atrogin-1 is a FoxO3 target gene that is typically increased during skeletal muscle atrophy and FoxO3 alone is able to increase atrogin-1 transcription. If miR-182 antagonizes FoxO3 expression, and thus its function, then miR-182 should prevent the Dex-induced increase in atrogin-1 mRNA. Increasing miR-182 in myotubes prevented an increase in atrogin-1 expression by Dex (FIG. 3A). Furthermore, increasing miR-182 also blocked the induction of LC3, ATG12, and cathepsin-L, three other FoxO3 targets associated with the autophagy/lysosome system (FIG. 3B-D)

miR-182 is Increased Exosomes Released from Myotubes Following Dex Administration

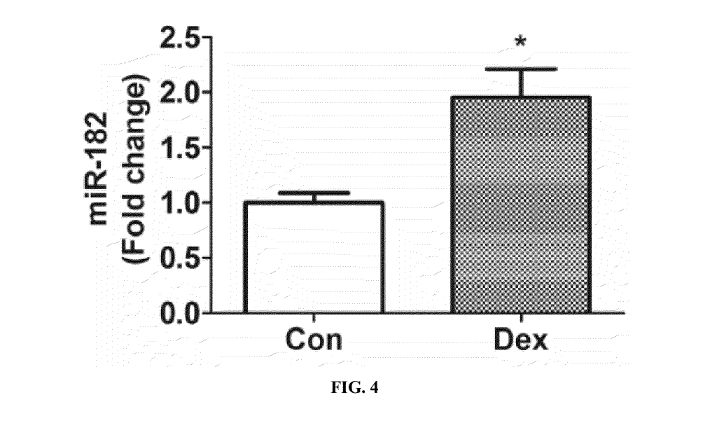

One mechanism by which microRNAs are protected from degradation is through incorporation into protective exosomes. This has led some to propose that the relative abundance patterns of microRNAs in various tissues and fluids as well as exosomes and other microvesicles are a reflection of underlying pathophysiological processes. Dex administration enhances the exosomal packaging and release of two atrophy-related microRNAs (miR-1 and miR-23a) into the media of C2C12 myotubes. This led us to test whether miR-182 is incorporated into exosomes that are released from myotubes. The amount of miR-182 in exosomes, normalized to U6 small nuclear RNA, is increased 95% in exosomes following Dex treatment to myotubes (FIG. 4).

### C2C12 Cell Culture

Mouse C2C12 myoblasts (American Type Culture Collection, Manassas, Va.) were cultured in growth media [Dulbecco modified Eagle medium plus 10% fetal bovine serum (Atlanta Biologicals, Lawrenceville, Ga.) and 1% penicillin and streptomycin (Invitrogen, Carlsbad, Calif.)] and studied between passage 3 and 7. Myoblasts were grown to ˜95% confluence in a 6-well plate and then differentiated into myotubes by replacing growth media with differentiation media (DMEM supplemented with 2% fetal bovine serum and 1% PS) for 3 days. In some experiments, myotubes were treated with 1 μM Dex for 6 hours before being harvested.

Transfection of microRNAs into myoblasts was performed when cells were ˜70% confluence using 40 nM miR-182 pre-miR or negative control pre-miR precursor (Ambion, Austin, Tex., USA) with Lipofectamine 2000 (Invitrogen, Carlsbad, Calif., USA) for 6 hours (9). Afterwards, cells were placed in differentiation media for 72 hours as described above before harvest for experiments. In some cases transfected myotubes were treated with 1 μM Dex for 6 hours before being harvested for analysis.

### Diabetes Model of Muscle Atrophy

Male rats (˜150 gm) received a single intravenous injection of STZ (125 mg/kg) in citrate buffer. STZ-injected and control rats were pair-fed and on the third day after injection the animals were anesthetized, and the gastrocnemius muscle was harvested. Food was withheld during the night before the tissue harvest to avoid the confounding influences of variable food intake on skeletal muscle protein metabolism. All animal studies were approved by the Emory Institutional Animal Care and Use Committee (IACUC).

Exosome Quantification and Exosome microRNA Analysis.

Exosomes are typically 40-100 nm membrane vesicles, which are secreted by most cell types. Exosomes can be found in saliva, blood, urine, amniotic fluid and malignant ascite fluids, among other biological fluids. As the RNA molecules encapsulated within exosomes are protected from degradation by RNAses they can be efficiently recovered from biological fluids, such as urine. Commercial kits for exosome isolation may be used to determine miR-182 contents. See Norgen's Urine Exosome RNA Isolation Kit. Users can simultaneously concentrate and isolate exosomal RNA, including microRNA, for use in assays.

For certain experiments, after several days (e.g. 3 days) in differentiation medium, cells were washed briefly with serum-free medium and then incubated in serum-free medium (control) with or without 1 μM Dex for 6 h. After 6 h the medium was collected. Medium was centrifuged at 800 g for 10 min to pellet intact cells and debris. The supernatant was centrifuged at 16,000 g for 60 min to pellet microvesicles, membrane particles, ectosomes, and apoptotic vesicles, which are larger than exosomes. The pellet was discarded, and the supernatant was centrifuged at 110,000 g for 1 h to pellet exosomes.

To quantify exosomal microRNA, exosome-associated RNA, including microRNA, was isolated using a urine exosome RNA isolation kit (Norgen Biotek, Thorolod, ON, Canada) according to the manufacturer's instructions for isolating RNA from extracellular fluids. Subsequent quantitative PCR (qPCR) was performed.

What we claim:

1. A method of evaluating a state of skeletal muscle atrophy comprising the step of measuring miR-182 in a sample from a subject wherein decreased quantities of miR-182 indicates an increased state of muscle atrophy in the subject.

2. The method of claim 1, wherein the sample is muscle, tissue, urine, urine exosome, blood, plasma, bodily fluid, or component thereof.

3. The method of claim 1, wherein the subject is a human subject.

4. The method of claim 1, wherein the subject is diagnosed, at risk of or exhibiting symptoms of cachexia, sepsis, chronic kidney disease, diabetes, renal failure, cancer, a chronic viral infection, HIV/AIDS, uremia, Dejerine Sottas syndrome, multiple sclerosis, tuberculosis, congestive heart failure, COPD, liver disease, muscular dystrophy, rheumatoid arthritis, amyotrophic lateral sclerosis (ALS), or other chronic or systemic disease.

5. The method of claim 1, wherein the subject is on a glucocorticoid therapy, mechanical ventilation, fasting, cast is put on a limb, extended bed rest or in a state of muscle disuse, or over 50, 60, or 65 years old.

6. The method of claim 1, wherein the method comprises the step of mixing the sample with a composition or surface comprising a probe that hybridizes to miR-182-5p and/or miR-182-3p and/or pre-miR-182 stem-loop and detecting hybridization of the probe to miR-182-5p and/or miR-182-3p and/or pre-miR-182 stem-loop in the sample under conditions such that an amount of miR-182-5p and/or miR-182-3p and/or pre-miR-182 stem-loop is quantified.

7. The method of claim 6, wherein the probe comprises a FRET acceptor and donor configured such that binding to the probe creates a light signal wherein measuring the intensity of the light signal indicates the quantity of miR-182-5p and/or miR-182-3p and/or pre-miR-182 stem-loop in the sample.

8. The method of claim 6, wherein the probe is radioactive further comprising the steps of measuring radioactivity and correlating radioactivity to the quantity of the probe that hybridizes to miR-182-5p and/or miR-182-3p and/or pre-miR-182 stem-loop in the sample.

9. The method of claim 6, wherein the probe is radioactive further comprising the step of mixing the composition with nucleases that specifically cleave single-stranded nucleic acids but do not cleave double-stranded nucleic acids.

10. The method of claim 1, wherein the amount of miR-182-5p and/or miR-182-3p and/or pre-miR-182 stem-loop is quantified by quantitative PCR.

11. The method of claim 6, wherein the surface comprises the probe conjugated to a surface.

12. The method of claim 11, wherein the surface is an array, bead, or nanoparticle.

13. A composition comprising a probe having a sequence of more than 7 or more nucleotides or nucleobases or continuous nucleotide nucleobases that is the reverse complement of SEQ ID NO: 1, 2, 3 or 4.

14. A surface comprising a probe having a sequence of more than 7 or more nucleotides or nucleobases or continuous nucleotide nucleobases that is the reverse complement of SEQ ID NO: 1, 2, 3 or 4.

15. A pharmaceutical composition comprising a nucleotide base comprising a nucleobase miR-182 and a pharmaceutically acceptable excipient.

16. The pharmaceutical composition of claim 15, wherein the nucleobase polymer is double stranded miR-182-5p.

17. The pharmaceutical composition of claim 15, wherein the nucleobase polymer comprises monomers of phosphodiester, phosphorothioate, methylphosphonate, phosphorodiamidate, piperazine phosphorodiamidate, ribose, 2′-O-methy ribose, 2′-O-methoxyethyl ribose, 2′-fluororibose, deoxyribose, 1-(hydroxymethyl)-2,5-dioxabicyclo[2.2.1]heptan-7-ol, P-(2-(hydroxymethyl)morpholino)-N,N-dimethylphosphonamidate, morpholin-2-ylmethanol, (2-(hydroxymethyl)morpholino) (piperazin-1-yl)phosphinate, or peptide nucleic acids or combinations thereof.

18. The pharmaceutical composition of claim 15 in the form of a pill, capsule, tablet, gel, or aqueous buffer comprising a saccharide.

19. A method of treating muscle wasting, cachexia or related disease or condition comprising administering an effective amount of a pharmaceutical composition of claim 15 to a subject in need thereof.

20. The method of claim 15, wherein the subject is diagnosed, at risk of or exhibiting symptoms of cachexia, sepsis, chronic kidney disease, diabetes, renal failure, cancer, a chronic viral infection, HIV/AIDS, uremia, Dejerine Sottas syndrome, multiple sclerosis, tuberculosis, congestive heart failure, COPD, liver disease, muscular dystrophy, rheumatoid arthritis, amyotrophic lateral sclerosis (ALS), or other chronic or systemic disease.
